(19) United States
(12) Patent Application Publication
(10) Pub. No.: US 2026/0337661 A1
(43) Pub. Date: Oct. 29, 2026

(54) BIOMARKERS OF THE INTEGRATED STRESS RESPONSE AND USES THEREOF

(71) Applicant: Calico Life Sciences LLC, South San Francisco, CA (US)

(72) Inventors: Amos Baruch, South San Francisco, CA (US); Ganesh Kolumam, South San Francisco, CA (US)

(21) Appl. No.: 19/465,356

(22) Filed: Jan. 30, 2026

Related U.S. Application Data
(63) Continuation of application No. PCT/US2024/040763, filed on Aug. 2, 2024.
(60) Provisional application No. 63/517,321, filed on Aug. 2, 2023.

(51) Int. Cl. C12Q 1/6809

(52) U.S. Cl. CPC C12Q 1/6809; C12Q 2600/106; C12Q 2600/136; C12Q 2600/158; G01N 2800/2814; G01N 2800/52

(57) ABSTRACT

The present disclosure provides compositions and methods useful in modulating integrated stress response (ISR) and associated diseases or disorders and methods of identifying ISR modulators. The present disclosure also provides methods of treating neurodegenerative conditions and predicting beneficial effects and identifying suitable targets for treatment.

### CROSS-REFERENCE TO RELATED APPLICATIONS

This application is a continuation of International (PCT) Patent Application No. PCT/US2024/040763, filed Aug. 2, 2024, which claims the benefit of, and priority to, U.S. Provisional Application No. 63/517,321, filed Aug. 2, 2023, the entire contents of each of which are incorporated herein by reference in their entirety.

### BACKGROUND

The integrated stress response (ISR) is a conserved mechanism that is activated following a wide variety of cellular insults. Induction of the ISR starts with the activation of one or more of 4 stress kinases that in turn phosphorylate the eukaryotic translation initiation factor 2a (eIF2α). Phosphorylated eIF2a (P-eIF2α) binds and inhibits eukaryotic translation initiation factor 2B (eIF2B), a hetero-pentameric guanine-exchange factor that is the rate limiting step in initiation of CAP-dependent translation. As a consequence, activation of the ISR suppresses global protein production within the cell. While cellular stress may be reversible, and, upon dephosphorylation of P-eIF2α, the cell would restore protein synthesis, a prolonged brake on protein synthesis resulting from maladaptive ISR perturbs cellular functions and initiates programmed cell death through the continuous expression of proapoptotic genes.

Loss of function mutations in any of the five eIF2B genes are associated with the autosomal recessive leukoencephalopathy vanishing white matter disease (VWMD). An acute cellular stress is typically followed by a recovery phase in which the dephosphorylation of P-eIF2α subunit by the ISR-induced phosphatase GADD34 would relieve eIF2B inhibition and restore protein synthesis. However, in VWMD, due to the genetically encoded eIF2B enzymatic deficiency, suppression of protein synthesis under stress is more pronounced and only partially restored once stress has been removed, resulting in condition in which the ISR is continuously activated. One of the most intriguing aspects of VWMD is that the elevated ISR in these patients is primarily confined to the central nervous system (CNS) with most symptoms deriving from spinal cord and brain deficiencies.

In addition to VWMD, dysregulation of the ISR (e.g., ISR activation) has been associated with numerous human diseases, including cancer and other diseases and disorders of the CNS, such as amyotrophic lateral sclerosis. There remains the need for identifying biomarkers suitable for monitoring ISR activation and attenuation specific to the CNS and guiding treatment strategies, predicting beneficial effects of treatments, and identifying targets for personalized treatment.

### SUMMARY

The invention is based, in part, upon the finding that under conditions in which the integrated stress response (ISR) is activated within the central nervous system, elevated GDF15 levels are present in the cerebrospinal fluid (CSF) but not when measured directly in plasma. Alternatively, such elevation in GDF15 upon ISR activation within the central nervous system can be detected in brain-derived extracellular vesicles. Related methods, such as treating neurodegenerative conditions and identifying suitable targets for treatment are also provided.

In one aspect, the present disclosure provides a method of treating a neurodegenerative condition, the method comprising: determining the GDF15 expression level in a sample obtained from a patient; comparing the GDF15 expression level in the sample to a reference; determining that the GDF15 expression level in the sample is elevated as compared to the reference; and administering an integrated stress response (ISR) modulator.

Also provided herein is a method of treating a neurodegenerative condition in a patient comprising administering to the patient an integrated stress response (ISR) modulator, wherein GDF15 expression level is elevated in a sample obtained from the patient prior to administration of the ISR modulator as compared to a reference.

Also provided herein is a method of treating a patient having integrated stress response dysregulation, the method comprising administering an integrated stress response (ISR) modulator to the patient, wherein GDF15 expression level in a sample obtained from the patient is elevated as compared to a reference.

Also provided herein is a method of evaluating a compound to determine whether the compound can modulate the integrated stress response, the method comprising: administering the compound to a subject; measuring GDF15 expression level in a sample obtained from the subject; and comparing the GDF15 expression level to a reference, wherein a significant difference between the GDF15 expression level and the reference is indicative that the compound can modulate the integrated stress response.

In some embodiments, the reference is determined by measuring the GDF15 expression level in a sample obtained from an aged match healthy subject. In some embodiments, the reference is determined by measuring the GDF15 expression level in a sample obtained from the subject before administering the compound.

In some embodiments, the sample is a fluid sample. In some embodiments, the fluid sample is a CSF sample. In some embodiments, the fluid sample is a blood sample. In some embodiments, the blood sample is enriched for central nervous system (CNS)-derived extracellular vesicles (EVs, e.g., exosomes). In some embodiments, the CNS-derived extracellular vesicles are astrocyte-derived exosomes (ADEs). In some embodiments, the CNS-derived extracellular vesicles are neuron-derived exosomes (NDEs).

In some embodiments, the GDF15 expression level is mRNA level or protein level. In some embodiments, the GDF15 expression level is significantly higher than the reference. In some embodiments, the GDF15 expression level is at least about 10%, at least about 20%, at least about 30%, at least about 40%, at least about 60%, or at least about 80% higher than the reference.

In some embodiments, the ISR modulator is an eIF2B activator. In some embodiments, the ISR modulator is a compound selected from Table 1, or a pharmaceutically acceptable salt thereof.

### BRIEF DESCRIPTION OF DRAWINGS

FIG. 1A depicts gene expression of GDF15 in the spinal cord and cerebellum of vanishing white matters (VWM) mice and wild-type (WT) mice (n=5 per group).

FIG. 1B and FIG. 1C depict GDF15 protein expression in cerebrospinal fluid (CSF) (FIG. 1B) and plasma (FIG. 1C) from WT and VWM mice collected at different ages. Values represented on a log 10 scale. Statistical significance was determined by comparing VWM to WT at the respective age using Mann Whitney test, *p<0.05, **p<0.01, ***p<0.001.

FIGS. 2A and 2B depict gene expression of the ISR signature and GDF15 in the cerebellum (FIG. 2A) and spinal cord (FIG. 2B) of VWM mice and WT mice following 4 months of treatment with 30 mg/kg 2BAct in food pellets or control chow. Samples were also collected after a drug washout period of 2 months.

FIG. 2C and FIG. 2D show GDF15 mRNA levels determined in cerebellum (FIG. 2C) and spinal cord (FIG. 2D) in WT mice, VWM mice, and VWM mice treated with 2BAct for 4 months and 2 months after cessation of 2BAct treatment. *p<0.05, **p<0.01, ***p<0.001 by Mann Whitney.

FIG. 2E and FIG. 2F show GDF15 protein levels determined in CSF (FIG. 2E) and plasma (FIG. 2F) in WT mice, VWM mice, and VWM mice treated with 2BAct for 4 months and 2 months after cessation of 2BAct treatment. *p<0.05, **p<0.01, ***p<0.001 by Mann Whitney.

FIG. 3A depicts the cell type annotation of single-cell RNA sequencing (scRNA-seq) analysis of forebrain from VWM mice.

FIG. 3B and FIG. 3C show the scRNA-seq expression of GDF15 and TRIB3, respectively, in forebrain from 2- and 5-month-old VWM mice and 5-months-old WT mice.

FIG. 4 shows GDF15 protein expression in CSF and plasma from healthy individuals and patients with VWMD. Box plots indicate median and interquartile range. Solid black shapes (□, •, ▪, ▾, ★) indicate longitudinal samples from the same VWMD patient. *p<0.05, **p<0.01, ***p<0.001 by one-way ANOVA.

FIG. 5A depicts gene expression of the ISR signature and GDF15 in the spinal cord of SOD1 G93A carrier mice as compared with WT B6 mice.

FIG. 5B depicts GDF15 protein expression in CSF and plasma from WT and SOD1 transgenic mice of the B6 or B6.SJL strains. Values represented on a log 10 scale. Statistical significance was determined by comparing SOD1 to WT at the respective background using Mann Whitney test, *p<0.05, **p<0.01, ***p<0.001. W=Weeks *p<0.05, **p<0.01, ***p<0.001.

FIG. 5C depicts gene expression of the ISR signature in the cerebellum of SOD1 G93A and WT mice.

FIG. 6A shows ISR signature is elevated in the spinal cord of ALS patients (n=104) as compared to healthy controls (n=22).

FIG. 6B depicts GDF15 protein expression determined in CSF and plasma from ALS patients, age-matched healthy controls (Ctrl(O)), or young healthy controls (Ctrl(Y)). Values represented on a log 10 scale. Statistical significance was determined using Mann Whitney test, *p<0.05, **p<0.01, ***p<0.001. W=Weeks *p<0.05, **p<0.01, ***p<0.001.

FIG. 6C depicts the correlation between astrocyte activation marker GFAP and GDF15 in ALS samples.

FIG. 7A shows that astrocyte derived exosomes (ADE) from VWM mice expressed 4.7-fold greater levels of GDF15 as compared to ADE from control mice. 2BAct downmodulated GDF15 expression on VWM ADE by 2.7-fold.

FIG. 7B shows that neuron derived exosomes (NDE) from VWM mice expressed 1.7-fold greater levels of GDF15 as compared to NDE from control mice. 2BAct downmodulated GDF15 expression on VWM NDE by 1.7-fold. Values represented on a log 10 scale. Statistical significance was determined using Mann Whitney test, *p<0.05, **p<0.01, ***p<0.001. W=Weeks *p<0.05, **p<0.01, ***p<0.001.

FIGS. 7C and 7D show ADEs (FIG. 7C) and NDEs (FIG. 7D) in plasma from VWM patients and compared to that of healthy subjects.

### DETAILED DESCRIPTION

### Definitions

As used herein, the singular forms “a,” “an,” and “the” include plural referents, unless the context clearly dictates otherwise. By way of example, “a compound” means one compound or more than one compound.

As used herein, the terms “about,” “approximately,” and “comparable to,” when used in reference to a value, refer to a value that is similar to the referenced value in the context of that referenced value. In general, those skilled in the art, familiar with the context, will appreciate the relevant degree of variance encompassed by “about,” “approximately,” and “comparable to” in that context. For example, in some embodiments, the terms “about,” “approximately,” and “comparable to” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

As used herein, the term “activation,” “activate,” “activating,” and the like in reference to a protein-activator (e.g., agonist) interaction means positively affecting (e.g., increasing) the activity or function of the protein (e.g., eIF2B, eIF2α, or component of the eIF2 or ISR pathway) relative to the activity or function of the protein in the absence of the activator. In some embodiments, activation refers to an increase in the activity of a signal transduction pathway or signaling pathway (e.g., eIF2B, eIF2α, or component of the eIF2 or ISR pathway). Thus, activation may include, at least in part, partially or totally increasing stimulation, increasing or enabling activation, or activating, sensitizing, or up-regulating signal transduction or enzymatic activity or the amount of a protein decreased in a disease (e.g., level of eIF2B, eIF2α, or component of the eIF2 or ISR pathway associated with a neurodegenerative disease). Activation may include, at least in part, partially or totally increasing stimulation, increasing or enabling activation, or activating, sensitizing, or up-regulating signal transduction or enzymatic activity or the amount of a protein (e.g., eIF2B, eIF2α, or component of the eIF2 or ISR pathway) that may modulate the level of another protein or increase cell survival (e.g., increase in eIF2α activity may increase cell survival in cells that may or may not have a reduction in eIF2α activity relative to a non-disease control).

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arterial, intradermal, subcutaneous, intraperitoneal, intraventricular, and intracranial. Other modes of delivery include, but are not limited to, the use of liposomal formulations, intravenous infusion, transdermal patches, etc.

As used herein, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

The term “associated” or “associated with” in the context of a substance or substance activity or function associated with a disease (e.g., a neurodegenerative disease) means that the disease is caused by (in whole or in part), or a symptom of the disease is caused by (in whole or in part) the substance or substance activity or function. For example, a symptom of a disease or condition associated with an impaired function of the eIF2B may be a symptom that results (entirely or partially) from a decrease in eIF2B activity (e.g. decrease in eIF2B activity or levels, increase in eIF2α phosphorylation or activity of phosphorylated eIF2α, or reduced eIF2 activity of the ISR signaling pathway). As used herein, what is described as being associated with a disease, if a causative agent, could be a target for treatment of the disease. For example, a disease associated with decreased eIF2 activity or eIF2 pathway activity, may be treated with an agent effective for increasing the level or activity of eIF2 or eIF2 pathway or a decrease in phosphorylated eIF2α activity or the ISR pathway. For example, a disease associated with phosphorylated eIF2α may be treated with an agent effective for decreasing the level of activity of phosphorylated eIF2α or a downstream component or effector of phosphorylated eIF2α. For example, a disease associated with eIF2α may be treated with an agent effective for increasing the level of activity of eIF2 or a downstream component or effector of eIF2.

The terms “disease”, “disorder”, and “condition” are used interchangeably herein.

As used herein, “eIF2B” refers to the heteropentameric eukaryotic translation initiation factor 2B. eIF2B is composed of five subunits: eIF2B1, eIF2B2, eIF2B3, eIF2B4, and eIF2B5. eIF2B1 refers to the protein associated with Entrez gene 1967, OMIM 606686, Uniprot Q14232, and/or RefSeq (protein) NP_001405. eIF2B2 refers to the protein associated with Entrez gene 8892, OMIM 606454, Uniprot P49770, and/or RefSeq (protein) NP_055054. eIF2B3 refers to the protein associated with Entrez gene 8891, OMIM 606273, Uniprot Q9NR50, and/or RefSeq (protein) NP_065098. eIF2B4 refers to the protein associated with Entrez gene 8890, OMIM 606687, Uniprot Q9UI10, and/or RefSeq (protein) NP_751945. eIF2B5 refers to the protein associated with Entrez gene 8893, OMIM 603945, Uniprot Q13144, and/or RefSeq (protein) NP_003898.

The terms “eIF2alpha,” “eIF2α,” and “eIF2α” are interchangeable and refer to the protein “eukaryotic translation initiation factor 2 alpha subunit eIF2S1.” In embodiments, “eIF2alpha,” “eIF2α,” and “eIF2α” refer to the human protein. Included in the terms “eIF2alpha,” “eIF2α,” and “eIF2α” are the wildtype and mutant forms of the protein. In some embodiments, “eIF2alpha,” “eIF2α,” or “eIF2α” refers to the protein associated with Entrez Gene 1965, OMIM 603907, UniProt P05198, and/or RefSeq (protein) NP_004085.

As used herein, the terms “elevation,” “elevated,” “upregulation,” and “upregulated,” are used interchangeably and, when used in connection with a cellular component, such as a gene or protein, refer to the increase of a quantity of the cellular component relative to a reference. Upregulation of a gene or protein refers to expression of the gene or protein that is higher than a selected reference. For instance, certain genes (e.g., GDF15) or protein (e.g., GDF15) in a patient having a neurodegenerative condition may be upregulated as compared to a healthy subject (e.g., an age-matched healthy subject). Similarly, the term “downregulation” or “downregulated” refers to the decrease of quantity of a cellular component, such as gene or protein, relative to a reference.

As used herein, an “effective amount” is an amount sufficient to accomplish a stated purpose. An example of an “effective amount” is an amount sufficient to alleviate to some extent one or more of the symptoms of the condition or disorder being treated when administered for treatment in a particular subject or subject population. In some embodiments, phrases “therapeutically effective amount” and “effective amount” are used interchangeably

As used herein, “GDF15” (“growth differentiation factor 15,” “macrophage inhibitory cytokine 1,” “MIC1,” “NAG-1,” “PTGFB,” “PLAB,” “prostate differentiation factor,” “non-steroidal anti-inflammatory drug-activated gene-1,” “NSAID-activated gene 1 protein,” “NSAID-regulated gene 1 protein,” “placental bone morphogenetic protein”) means human GDF15 defined by UniProt Q99988, a fragment thereof, or a variant thereof. In some embodiments, GDF15 refers to the protein associated with Entrez Gene 9518, OMIM 605312, and/or RefSeq (protein) NP_004855.2. In some particular embodiments, GDF15 of non-human species, e.g., mouse GDF15, is used.

As used herein, the term “inhibition,” “inhibit,” “inhibiting,” and the like in reference to a protein-inhibitor (e.g., antagonist) interaction means negatively affecting (e.g., decreasing) the activity or function of the protein relative to the activity or function of the protein in the absence of the inhibitor. In some embodiments, inhibition refers to reduction of a disease or symptoms of disease. In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein. In some embodiments, inhibition refers to a decrease in the activity of a signal transduction pathway or signaling pathway (e.g., eIF2B, eIF2α, or a component of the eIF2 pathway, pathway activated by eIF2α phosphorylation, or ISR pathway). Thus, inhibition may include, at least in part, partially or totally decreasing stimulation, decreasing or reducing activation, or inactivating, desensitizing, or down-regulating signal transduction or enzymatic activity or the amount of a protein increased in a disease (e.g. eIF2B, eIF2α, or a component of the eIF2 or ISR pathway, wherein each is associated with, e.g., a neurodegenerative disease). Inhibition may include, at least in part, partially or totally decreasing stimulation, decreasing or reducing activation, or deactivating, desensitizing, or down-regulating signal transduction or enzymatic activity or the amount of a protein (e.g. eIF2B, eIF2α, or component of the eIF2 or ISR pathway) that may modulate the level of another protein or increase cell survival (e.g., decrease in phosphorylated eIF2α pathway activity may increase cell survival in cells that may or may not have an increase in phosphorylated eIF2α pathway activity relative to a non-disease control or decrease in eIF2α pathway activity may increase cell survival in cells that may or may not have an increase in eIF2α pathway activity relative to a non-disease control).

As used herein, the term “modulation” refers to an increase or decrease in the level of a target molecule or the function of a target molecule. In some embodiments, modulation of eIF2B, eIF2α, or a component of the eIF2 or ISR pathway may result in reduction of the severity of one or more symptoms of a disease associated with eIF2B, eIF2α, or a component of the eIF2 or ISR pathway (e.g., a neurodegenerative condition) or a disease that is not caused by eIF2B, eIF2α, or a component of the eIF2 pathway or ISR pathway but may benefit from modulation of eIF2B, eIF2α, or a component of the eIF2 or ISR pathway. The term “modulator” refers to modulation of (e.g., an increase or decrease in) the level of a target molecule or the function of a target molecule. In embodiments, a modulator of eIF2B, eIF2α, or component of the eIF2 or ISR pathway is a neuroprotectant.

The term “optional” or “optionally” means that the subsequently described event or circumstance can or cannot occur, and that the description includes instances where the event or circumstance occurs and instances where it does not.

As used herein, a “patient” refers to any animal suffering from or diagnosed with a disease, disorder, or condition, such as a neurodegenerative condition, including, but not limited to, mammals, primates, and humans. In certain embodiments, the patient may be a non-human mammal such as, for example, a cat, a dog, or a horse. In a preferred embodiment, the patient is a human subject.

As used herein, “pharmaceutically acceptable excipient” or “pharmaceutically acceptable carrier” refers to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “signaling pathway” or “signal transduction pathway,” as used herein, refers to a series of interactions between cellular and optionally extracellular components (e.g., proteins, nucleic acids, small molecules, ions, lipids) that conveys a change in one component to one or more other components, which in turn may convey a change to additional components, which is optionally propagated to other signaling pathway components.

As used herein, a “subject” refers to an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate (e.g., cynomolgus monkey or rhesus monkey or human). In embodiments, the subject is a human. The terms “subject” and “patient” may be used interchangeably; however, in some embodiments, a subject may not be diagnosed with or suffering from a disease, disorder, or condition, but may be in need of treatment by a method described herein. In some embodiments, a patient or a subject may be a participant of a clinical study.

As used herein, “treat,” “treating,” or “treatment” refers to a method of alleviating or abrogating a disease and/or its attendant symptoms.

As used herein, “pharmaceutically acceptable salt” refers to those salts which retain the biological effectiveness and properties of the free bases and which are obtained by reaction with inorganic acids, for example, hydrochloric acid, hydrobromic acid, sulfuric acid, nitric acid, and phosphoric acid or organic acids such as sulfonic acid, carboxylic acid, organic phosphoric acid, methanesulfonic acid, ethanesulfonic acid, p-toluenesulfonic acid, citric acid, fumaric acid, maleic acid, succinic acid, benzoic acid, salicylic acid, lactic acid, mono-malic acid, mono oxalic acid, tartaric acid such as mono tartaric acid (e.g., (+) or (−)-tartaric acid or mixtures thereof), amino acids (e.g., (+) or (−)-amino acids or mixtures thereof), and the like. These salts can be prepared by methods known to those skilled in the art.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972).

Unless otherwise defined, all technical and scientific terms used herein have the same meaning as commonly understood by one of ordinary skill in the art to which this disclosure belongs.

### Integrated Stress Response Biomarker

The present disclosure is based in part on the discovery of GDF15 as biomarker that is indicative for integrated stress response (ISR) activation in the central nervous system (CNS). Accordingly, GDF15 can be used as a biomarker to diagnose disease, monitor disease progression, guide treatment strategies, and predict treatment response of neurodegenerative conditions associated with ISR.

In some embodiments, GDF15 expression is elevated in the CNS of a subject under ISR activation, associated with a neurodegenerative condition. In some embodiments, GDF15 expression is elevated in a sample obtained from the subject relative to a reference. In some embodiments, the sample is a CSF sample. In some embodiments, the sample is a blood sample. In embodiments, the blood sample may be optionally enriched for CNS-derived (e.g., astrocyte-derived or brain-derived) extracellular vesicles (EVs) (e.g., exosomes). In some embodiments, GDF15 expression is elevated in a CSF sample or a blood sample enriched for CNS-derived EVs (e.g., astrocyte-derived EVs or neuron-derived EVs) more significantly than in the whole blood or plasma of the subject. In some embodiments, GDF15 expression is essentially not elevated in the whole blood or plasma of the subject relative to a reference.

The GDF15 expression level may be determined by methods known in the art for measuring the GDF15 gene (e.g., mRNA) expression or GDF15 protein expression. For example, GDF15 gene expression level may be quantified by, e.g., real-time PCR (quantitative PCR, qPCR), microarray, next-generation sequencing (RNA-Seq), northern blotting, and serial analysis of gene expression (SAGE); GDF15 protein expression level may be quantified by, e.g., immunohistochemistry (IHC), enzyme-linked immunosorbent assay (ELISA), protein immunoprecipitation, immunoelectrophoresis, western blot, protein immunostaining, and spectrometry methods (e.g., chromatography, mass spectrometry).

To determine whether GDF15 expression is elevated in a sample obtained from the subject, the expression level of GDF15 may be compared to a reference. In some embodiments, the reference may be determined by measuring the expression level of GDF15 in a different sample from the same subject. In some other embodiments, the reference may be determined by measuring the expression level of GDF15 in a sample of the same type obtained from a healthy subject, e.g., an age-matched healthy subject. In some embodiments, the GDF15 expression level is at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or greater than about 90% higher than the GDF15 expression level of the reference.

In some embodiments, the elevation of GDF15 expression is statistically significant as compared with the reference. In some embodiments, the elevation of the GDF15 expression level in the CNS (e.g., as present in the CSF or in CNS-derived exosomes) of a patient having a neurodegenerative condition as compared to that in the whole blood or plasma of the same patient is statistically significant. In some embodiments, the elevation of the GDF15 expression level in the CNS (e.g., as present in the CSF or CNS-derived exosomes) of a patient having a neurodegenerative condition as compared to that of a healthy subject (e.g., an age-matched healthy subject) is statistically significant. In some embodiments, statistical significance may be determined using a standard statistical test, for example, Student's T Test or Mann-Whitney U test. In some embodiments, the neurodegenerative condition is associated with ISR dysregulation.

### Methods of Treatment and Identifying Patients for Treatment

Provided herein are methods of treating neurodegenerative conditions associated with ISR dysregulation. Particularly, methods are provided for treating a patient having a neurodegenerative condition with an eIF2B activator.

In some embodiments, the present disclosure provides a method of detecting a patient having ISR dysregulation. In some embodiments, the method comprises detecting an elevation in expression of GDF15 in a sample (e.g., a CSF sample or a blood sample enriched for CNS-derived EVs, e.g., astrocyte-derived EVs or neuron-derived EVs) obtained from the patient as compared to a reference, wherein the elevation in expression indicates an ISR dysregulation. Also provided herein are methods of treating a patient having a neurodegenerative condition, the method comprising administering to the patient an eIF2B activator, when GDF15 expression level is elevated in a sample (e.g., a CSF sample or a blood sample enriched for CNS-derived EVs, e.g., astrocyte-derived EVs or neuron-derived EVs) from the patient as compared to a reference. In some embodiments, administration of an eIF2B activator to the patient is capable of suppressing GDF15 elevation in the patient, e.g., GDF15 elevation in a CSF sample or a blood sample enriched for CNS-derived EVs (e.g., astrocyte-derived EVs or neuron-derived EVs).

In some embodiments, the neurodegenerative condition may be a leukodystrophy, a leukoencephalopathy, a hypomyelinating or demyelinating disease, an intellectual disability syndrome (e.g., fragile X syndrome), a cognitive impairment, a glial cell dysfunction, or a brain injury (e.g., a traumatic brain injury, hypoxia induced brain injury or toxin induced brain injury). In some embodiments, the neurodegenerative condition may be vanishing white matter disease, childhood ataxia with CNS hypo myelination, Alzheimer's disease, amyotrophic lateral sclerosis (ALS), Creutzfeldt-Jakob disease, frontotemporal dementia (FTD), Gerstmann-Straussler-Scheinker disease, Huntington's disease, dementia (e.g., HIV-associated dementia or Lewy body dementia), kuru, multiple sclerosis, Parkinson's disease, or a prion disease.

In some embodiments, the reference of a provided method may be determined by measuring the expression level of GDF15 in a different sample from the same patient or in a sample of the same type from a healthy subject (e.g., an age-matched healthy subject).

### Methods of Evaluating a Compound for its Ability to Modulate the ISR

The present disclosure also provides methods of evaluating a compound to determine whether the compound can modulate the ISR signaling pathway. In some embodiments, provided herein is a method of evaluating a compound to determine whether the compound can activate eIF2B. In some embodiments, a provided method comprises:
(a) administering the compound to a subject;
(b) measuring GDF15 expression level in a sample (e.g., a CSF sample or a blood sample enriched for CNS-derived EVs, e.g., astrocyte-derived EVs or neuron-derived EVs) obtained from the subject;
(c) comparing the GDF15 expression level to a reference, wherein a significant difference between the GDF15 expression level and the reference is indicative that the compound can modulate the integrated stress response. The reference may be an GDF15 expression level measured in a sample of the same type from an untreated subject.

In some embodiments, a compound evaluated by the provided method modulates one or more components in the ISR pathway, and the GDF15 expression level is decreased in the cerebrospinal fluid sample, astrocyte-derived EVs, or neuron-derived EVs as compared to the reference. In some embodiments, the GDF15 expression level is decreased by about 5% or more, about 10% or more, about 15% or more, about 20% or more, about 25% or more, about 30% or more, about 35% or more, about 40% or more, about 45% or more, about 50% or more, about 60% or more, about 70% or more, about 80% or more, or about 90% or more in the cerebrospinal fluid sample, astrocyte-derived EVs, or neuron-derived EVs as compared to the reference.

### ISR Modulators of the Disclosure

In some embodiments, a compound for treatment of neurodegenerative conditions, or manifestation or symptoms thereof, of the disclosure may modulate the ISR signaling pathway. In some embodiments, the ISR signaling pathway modulator is an eIF2B activator.

In some embodiments, the ISR signaling pathway modulator is a compound described in international patent applications PCT/US2017/031367 and PCT/US2019/055850, incorporated herein by reference in their entirety.

In some embodiments, the ISR signaling pathway modulator is a compound represented by Formula (I):

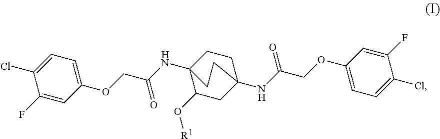

or a pharmaceutically acceptable salt thereof, wherein:
R¹ is selected from the group consisting of —C(O)—C₁₋₄alkyl, —C(O)—O—C₁₋₄alkyl, —C(O)—N(R^{a})—C₁₋₄alkyl, —C(O)—C₁₋₄alkylene-C₁₋₄alkoxy, —C(O)—C₁₋₄alkylene-O—C₁₋₄alkylene-C₁₋₄alkoxy, -methylene-O—P(O)(OH)₂, —C(O)—C₁₋₅alkylene-O—P(O)(OH)₂, —C(O)—O—C₁₋₅alkylene-O—P(O)(OH)₂, —C(O)—N(R^{a})—C₁₋₅alkylene-O—P(O)(OH)₂, —C(O)—C₁₋₅alkylene-P(O)(OH)₂, —C(O)—C₁₋₅alkylene-phenylene-O—P(O)(OH)₂, —C(O)—C₁₋₅alkylene-phenylene-(O—P(O)(OH)₂)₂, —C(O)—C₁₋₅alkylene-phenylene-(O—P(O)(OH)₂)(O—C₁₋₅alkylene-P(O)(OH)₂), -methylene-O—C(O)C₁₋₅alkylene-phenylene-O—P(O)(OH)₂, -methylene-O—C(O)C₁₋₅alkylene-phenylene-(O—P(O)(OH)₂)₂, —C(O)—O—C₁₋₅alkylene-O—C(O)C₁₋₅alkylene-phenylene-O—P(O)(OH)₂, —C(O)—N(R^{a})-heteroarylene-C₁₋₂alkylene-O—P(O)(OH)₂, —P(O)(OH)₂, —SO₃H, —SO₂NR^{a}R^{b}, —C(O)-heteroaryl, —C(O)—C₁₋₅alkylene-O—C₁₋₅alkylene-phenyl and methylene-C₁₋₅alkoxide;
wherein —C(O)—C₁₋₄alkyl is substituted by one or two substituents each independently selected from the group consisting of —NR^{a}R^{b} and —CO₂H; and wherein —C(O)—O—C₁₋₄alkyl, —C(O)—N(R^{a})—C₁₋₄alkyl, —C(O)—C₁₋₄alkylene-C₁₋₄alkoxy, —C(O)—C₁₋₄alkylene-O—C₁₋₄alkylene-C₁₋₄alkoxy, methylene-O—P(O)(OH)₂, —C(O)—C₁₋₅alkylene-O—P(O)(OH)₂, —C(O)—O—C₁₋₅alkylene-O—P(O)(OH)₂, —C(O)—N(R^{a})—C₁₋₅alkylene-O—P(O)(OH)₂, —C(O)—C₁₋₅alkylene-P(O)(OH)₂, —C(O)—C₁₋₅alkylene-phenylene-O—P(O)(OH)₂, —C(O)—C₁₋₅alkylene-phenylene-(O—P(O)(OH)₂)₂, —C(O)—C₁₋₅alkylene-phenylene-(O—P(O)(OH)₂)(O—C₁₋₅alkylene-P(O)(OH)₂), -methylene-O—C(O)C₁₋₅alkylene-phenylene-O—P(O)(OH)₂, -methylene-O—C(O)C₁₋₅alkylene-phenylene-(O—P(O)(OH)₂)₂, —C(O)—O—C₁₋₅alkylene-O—C(O)C₁₋₅alkylene-phenylene-O—P(O)(OH)₂, —C(O)-heteroaryl, —C(O)—C₁₋₅alkylene-O—C₁₋₅alkylene-phenyl and —C(O)—N(R^{a})-heteroarylene-C₁₋₂alkylene-O—P(O)(OH)₂ may optionally be substituted by one, two, three or four substituents each independently selected from the group consisting of halogen, —CO₂H, —NR^{a}R^{b} and C₁₋₂alkyl (optionally substituted by one, two or three fluorines) and aryl; and
R^{a} and R^{b} are independently selected, for each occurrence, from the group consisting of hydrogen and C₁₋₃alkyl.

In some embodiments, R¹ is selected from the group consisting of

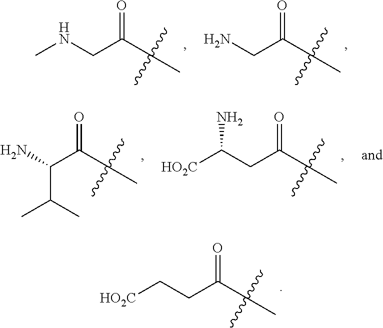

In some embodiments, R¹ is —C(O)—O—C₁₋₄alkyl. In some embodiments, R¹ is selected from the group consisting of

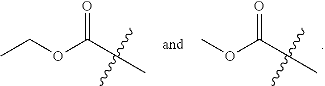

In some embodiments, R¹ is —C(O)—N(R^{a})—C₁₋₄alkyl, wherein —C(O)—N(R^{a})—C₁₋₄alkyl may optionally be substituted by one or two —CO₂H groups. In some embodiments, R¹ is selected from the group consisting of

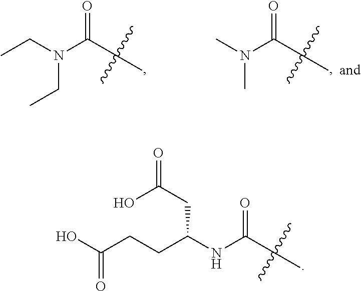

In some embodiments, R¹ is —C(O)—C₁₋₄alkylene-C₁₋₄alkoxy. In some embodiments, R¹ is selected from the group consisting of

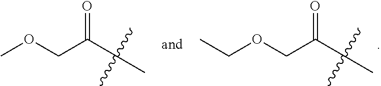

In some embodiments, R¹ is —C(O)—C₁₋₄alkylene-O—C₁₋₄alkylene-C₁₋₄alkoxy. In some embodiments, R¹ is represented by

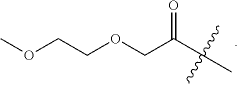 [SMILES: *C(=O)COCCOC]

In some embodiments, R¹ is -methylene-O—P(O)(OH)₂. In some embodiments, R¹ is represented by

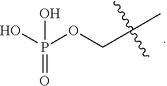 [SMILES: *COP(=O)(O)O]

In some embodiments, R¹ is —C(O)—C₁₋₅alkylene-O—P(O)(OH)₂. In some embodiments, R¹ is selected from the group consisting of

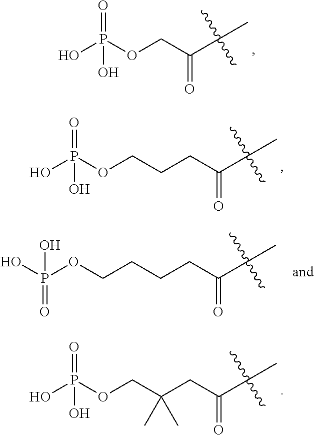

In some embodiments, R¹ is —C(O)—O—C₁₋₅alkylene-O—P(O)(OH)₂. In some embodiments, R¹ is selected from the group consisting of

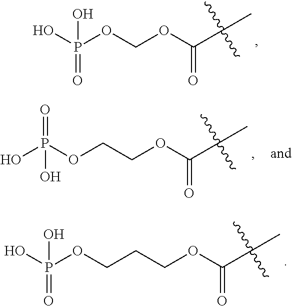

In some embodiments, R¹ is —C(O)—N(R^{a})—C₁₋₅alkylene-O—P(O)(OH)₂. In some embodiments, R¹ is selected from the group consisting of

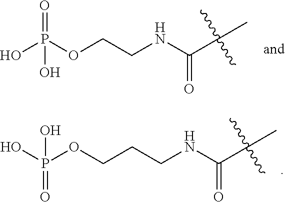 [SMILES: *C(=O)NCCOP(=O)(O)O ; *C(=O)NCCCOP(=O)(O)O]

In some embodiments, R¹ is —C(O)—C₁₋₅alkylene-P(O)(OH)₂. In some embodiments, R¹ is represented by

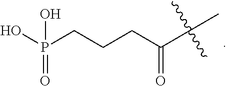 [SMILES: *C(=O)CCCP(=O)(O)O]

In some embodiments, R¹ is —C(O)—C₁₋₅alkylene-phenylene-O—P(O)(OH)₂, —C(O)—C₁₋₅alkylene-phenylene-(O—P(O)(OH)₂)₂ or —C(O)—C₁₋₅alkylene-phenylene-(O—P(O)(OH)₂)(O—C₁₋₅alkylene-P(O)(OH)₂). In some embodiments, R¹ is selected from the group consisting of

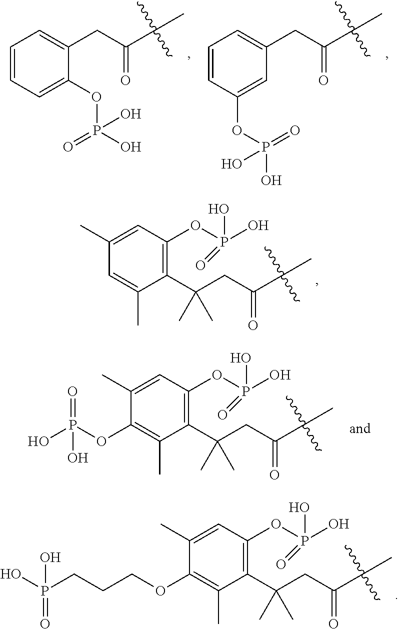

In some embodiments, R¹ is —C(O)—N(R^{a})-heteroarylene-C₁₋₂alkylene-O—P(O)(OH)₂. In some embodiments, R¹ is represented by

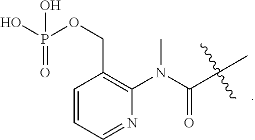 [SMILES: *C(=O)N(C)c1ncccc1COP(=O)(O)O]

In some embodiments, R¹ is —C(O)-heteroaryl. In some embodiments, R¹ is selected from the group consisting of

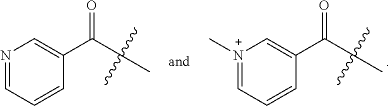

In some embodiments, R¹ is —C(O)—C₁₋₅alkylene-O—C₁₋₅alkylene-phenyl. In some embodiments, R¹ is represented by

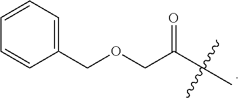 [SMILES: *C(=O)COCc1ccccc1]

In some embodiments, R¹ is methylene-C₁₋₅alkoxide. In some embodiments, R¹ is represented by

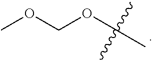 [SMILES: *OCOC]

In some embodiments, R¹ is —C(O)—O—C₁₋₅alkylene-O—C(O)C₁₋₅alkylene-phenylene-O—P(O)(OH)₂, -methylene-O—C(O)C₁₋₅alkylene-phenylene-O—P(O)(OH)₂ or -methylene-O—C(O)C₁₋₅alkylene-phenylene-(O—P(O)(OH)₂)₂. In some embodiments, R¹ is selected from the group consisting of

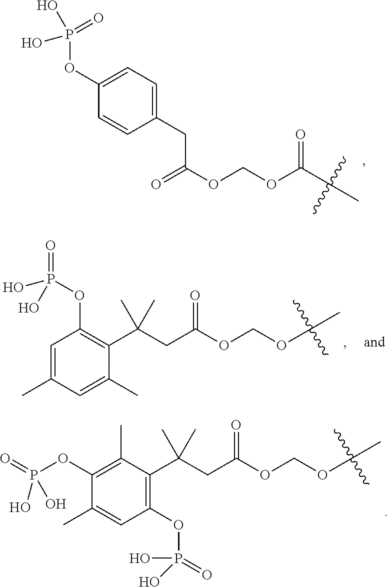

In some embodiments, R¹ is selected from the group consisting of —P(O)(OH)₂, —SO₃H, and —SO₂NH₂.

In some embodiments, the eIF2B activator is a compound set forth in Table 1 or a pharmaceutically acceptable salt thereof.

TABLE 1
Exemplary compounds of the invention

| Compound | |
|---|---|
| Number | Structure |
| 100 | 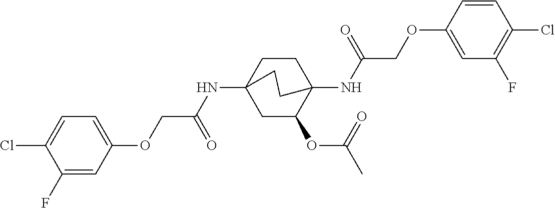 [SMILES: CC(=O)O[C@H]1CC2(NC(=O)COc3ccc(Cl)c(F)c3)CCC1(NC(=O)COc1ccc(Cl)c(F)c1)CC2] |
| 101 | 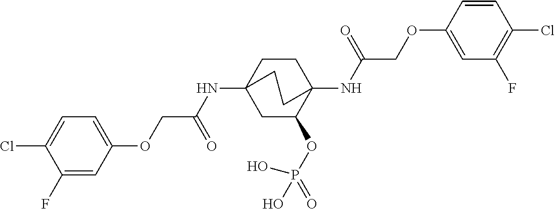 [SMILES: O=C(COc1ccc(Cl)c(F)c1)NC12CCC(NC(=O)COc3ccc(Cl)c(F)c3)(CC1)[C@@H](OP(=O)(O)O)C2] |
| 102 | 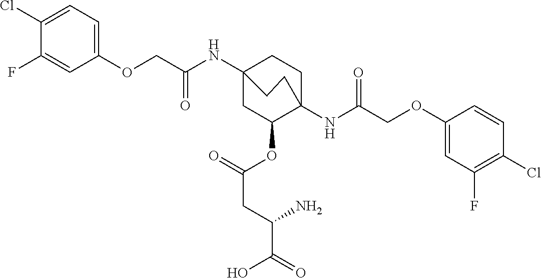 [SMILES: N[C@@H](CC(=O)O[C@H]1CC2(NC(=O)COc3ccc(Cl)c(F)c3)CCC1(NC(=O)COc1ccc(Cl)c(F)c1)CC2)C(=O)O] |
| 103 | 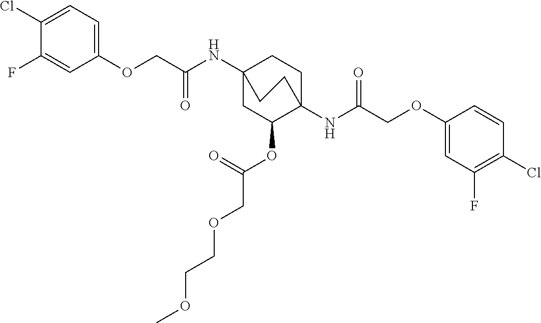 [SMILES: COCCOCC(=O)O[C@H]1CC2(NC(=O)COc3ccc(Cl)c(F)c3)CCC1(NC(=O)COc1ccc(Cl)c(F)c1)CC2] |
| 104 | 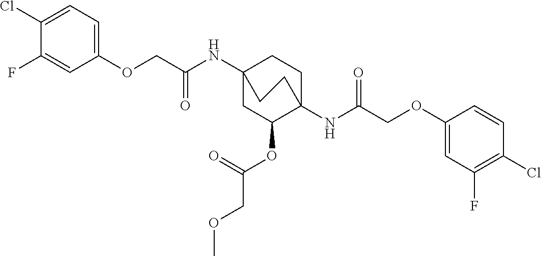 [SMILES: COCC(=O)O[C@H]1CC2(NC(=O)COc3ccc(Cl)c(F)c3)CCC1(NC(=O)COc1ccc(Cl)c(F)c1)CC2] |
| 105 | 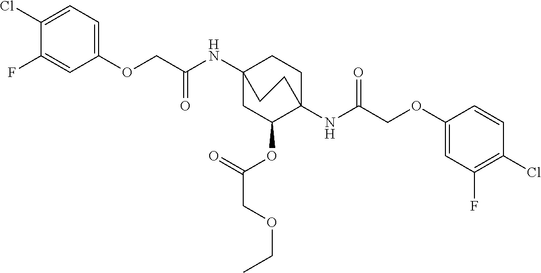 [SMILES: CCOCC(=O)O[C@H]1CC2(NC(=O)COc3ccc(Cl)c(F)c3)CCC1(NC(=O)COc1ccc(Cl)c(F)c1)CC2] |
| 106 | 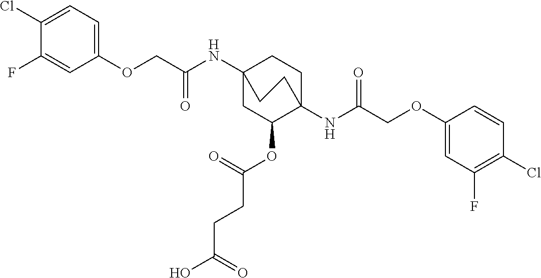 [SMILES: O=C(O)CCC(=O)O[C@H]1CC2(NC(=O)COc3ccc(Cl)c(F)c3)CCC1(NC(=O)COc1ccc(Cl)c(F)c1)CC2] |
| 107 | 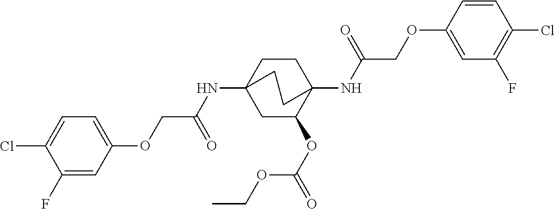 [SMILES: CCOC(=O)O[C@H]1CC2(NC(=O)COc3ccc(Cl)c(F)c3)CCC1(NC(=O)COc1ccc(Cl)c(F)c1)CC2] |
| 108 | 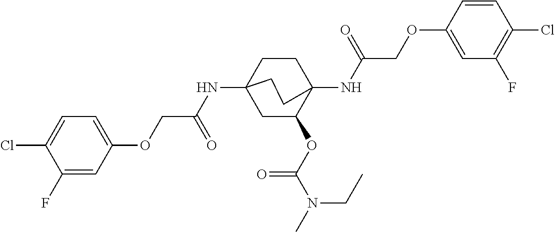 [SMILES: CCN(C)C(=O)O[C@H]1CC2(NC(=O)COc3ccc(Cl)c(F)c3)CCC1(NC(=O)COc1ccc(Cl)c(F)c1)CC2] |
| 109 | 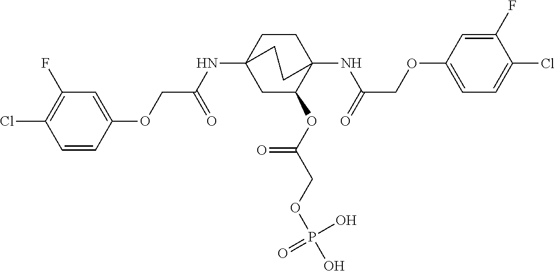 [SMILES: O=C(COc1ccc(Cl)c(F)c1)NC12CCC(NC(=O)COc3ccc(Cl)c(F)c3)(CC1)[C@@H](OC(=O)COP(=O)(O)O)C2] |
| 110 | 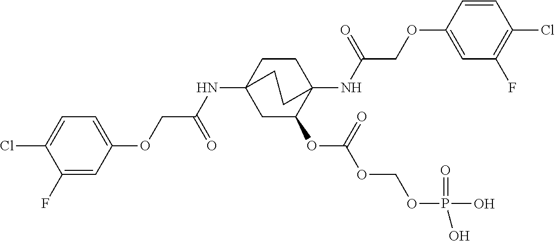 [SMILES: O=C(COc1ccc(Cl)c(F)c1)NC12CCC(NC(=O)COc3ccc(Cl)c(F)c3)(CC1)[C@@H](OC(=O)OCOP(=O)(O)O)C2] |
| 111 | 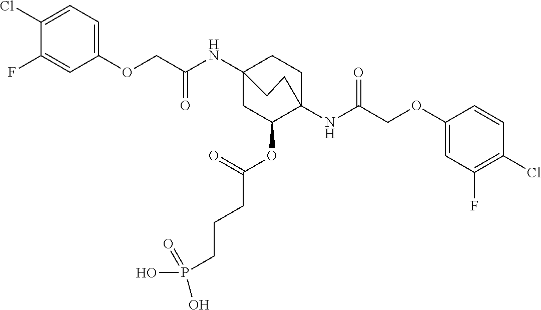 [SMILES: O=C(COc1ccc(Cl)c(F)c1)NC12CCC(NC(=O)COc3ccc(Cl)c(F)c3)(CC1)[C@@H](OC(=O)CCCP(=O)(O)O)C2] |
| 112 | 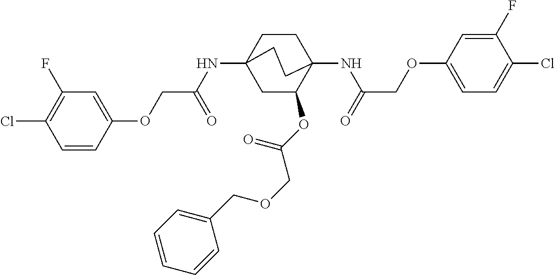 [SMILES: O=C(COc1ccc(Cl)c(F)c1)NC12CCC(NC(=O)COc3ccc(Cl)c(F)c3)(CC1)[C@@H](OC(=O)COCc1ccccc1)C2] |
| 113 | 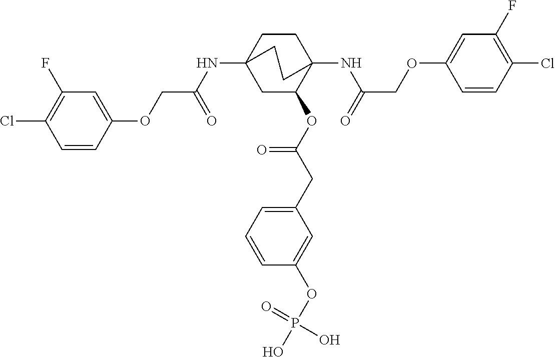 [SMILES: O=C(COc1ccc(Cl)c(F)c1)NC12CCC(NC(=O)COc3ccc(Cl)c(F)c3)(CC1)[C@@H](OC(=O)Cc1cccc(OP(=O)(O)O)c1)C2] |
| 114 | 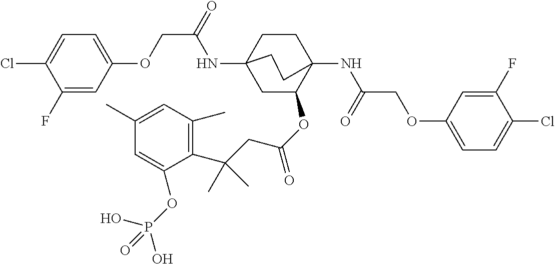 [SMILES: Cc1cc(C)c(C(C)(C)CC(=O)O[C@H]2CC3(NC(=O)COc4ccc(Cl)c(F)c4)CCC2(NC(=O)COc2ccc(Cl)c(F)c2)CC3)c(OP(=O)(O)O)c1] |
| 115 | 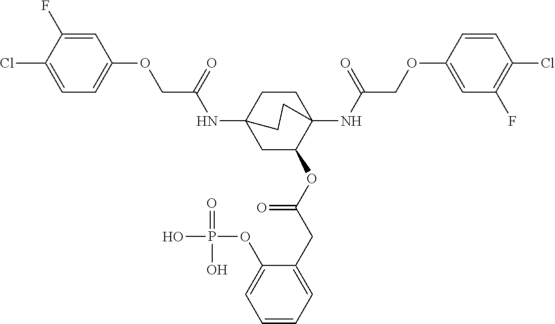 [SMILES: O=C(COc1ccc(Cl)c(F)c1)NC12CCC(NC(=O)COc3ccc(Cl)c(F)c3)(CC1)[C@@H](OC(=O)Cc1ccccc1OP(=O)(O)O)C2] |
| 116 | 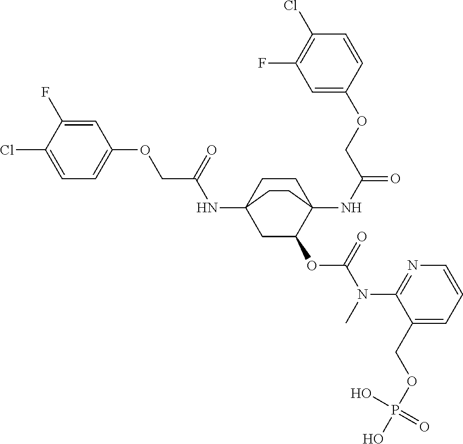 [SMILES: CN(C(=O)O[C@H]1CC2(NC(=O)COc3ccc(Cl)c(F)c3)CCC1(NC(=O)COc1ccc(Cl)c(F)c1)CC2)c1ncccc1COP(=O)(O)O] |
| 117 | 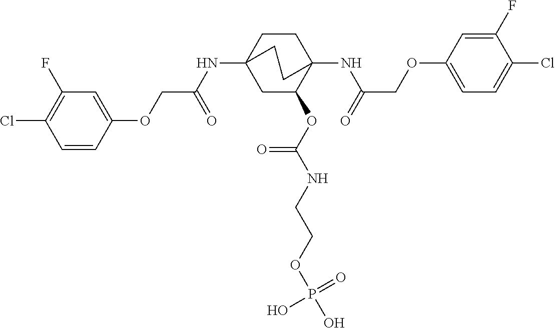 [SMILES: O=C(COc1ccc(Cl)c(F)c1)NC12CCC(NC(=O)COc3ccc(Cl)c(F)c3)(CC1)[C@@H](OC(=O)NCCOP(=O)(O)O)C2] |
| 118 | 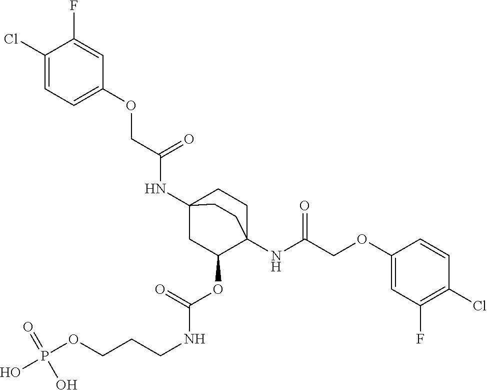 [SMILES: O=C(COc1ccc(Cl)c(F)c1)NC12CCC(NC(=O)COc3ccc(Cl)c(F)c3)(CC1)[C@@H](OC(=O)NCCCOP(=O)(O)O)C2] |
| 119 | 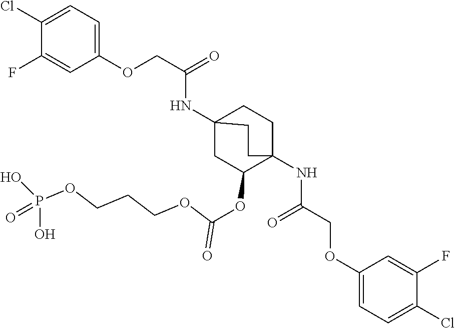 [SMILES: O=C(COc1ccc(Cl)c(F)c1)NC12CCC(NC(=O)COc3ccc(Cl)c(F)c3)(CC1)[C@@H](OC(=O)OCCCOP(=O)(O)O)C2] |
| 120 | 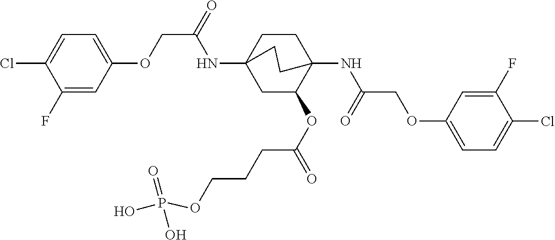 [SMILES: O=C(COc1ccc(Cl)c(F)c1)NC12CCC(NC(=O)COc3ccc(Cl)c(F)c3)(CC1)[C@@H](OC(=O)CCCOP(=O)(O)O)C2] |
| 121 | 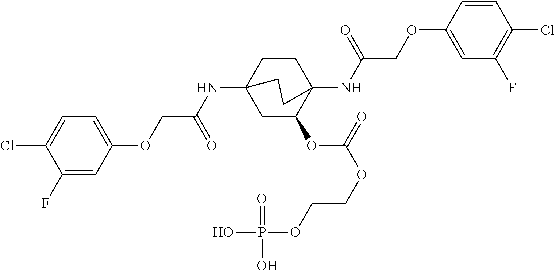 [SMILES: O=C(COc1ccc(Cl)c(F)c1)NC12CCC(NC(=O)COc3ccc(Cl)c(F)c3)(CC1)[C@@H](OC(=O)OCCOP(=O)(O)O)C2] |
| 122 | 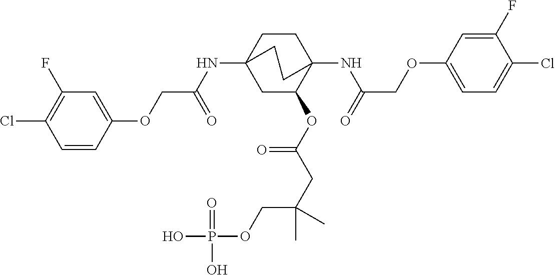 [SMILES: CC(C)(COP(=O)(O)O)CC(=O)O[C@H]1CC2(NC(=O)COc3ccc(Cl)c(F)c3)CCC1(NC(=O)COc1ccc(Cl)c(F)c1)CC2] |
| 123 | 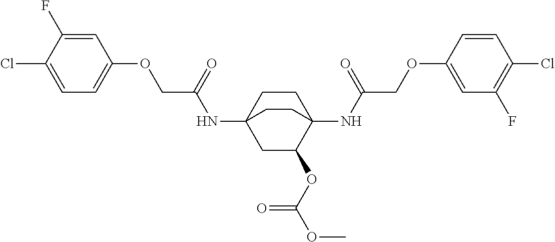 [SMILES: COC(=O)O[C@H]1CC2(NC(=O)COc3ccc(Cl)c(F)c3)CCC1(NC(=O)COc1ccc(Cl)c(F)c1)CC2] |
| 124 | 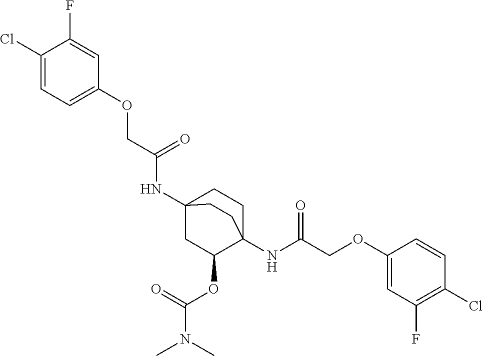 [SMILES: CN(C)C(=O)O[C@H]1CC2(NC(=O)COc3ccc(Cl)c(F)c3)CCC1(NC(=O)COc1ccc(Cl)c(F)c1)CC2] |
| 125 | 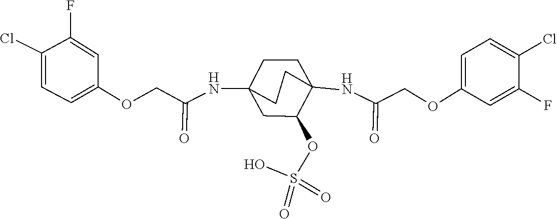 [SMILES: O=C(COc1ccc(Cl)c(F)c1)NC12CCC(NC(=O)COc3ccc(Cl)c(F)c3)(CC1)[C@@H](OS(=O)(=O)O)C2] |
| 126 | 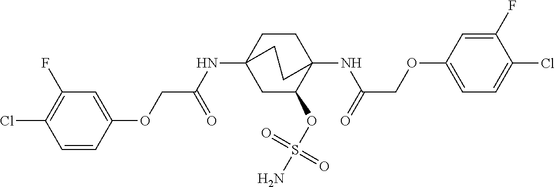 [SMILES: NS(=O)(=O)O[C@H]1CC2(NC(=O)COc3ccc(Cl)c(F)c3)CCC1(NC(=O)COc1ccc(Cl)c(F)c1)CC2] |
| 127 | 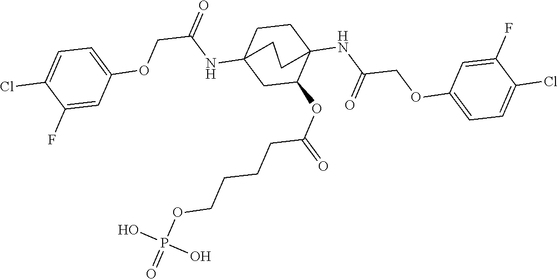 [SMILES: O=C(COc1ccc(Cl)c(F)c1)NC12CCC(NC(=O)COc3ccc(Cl)c(F)c3)(CC1)[C@@H](OC(=O)CCCCOP(=O)(O)O)C2] |
| 128 | 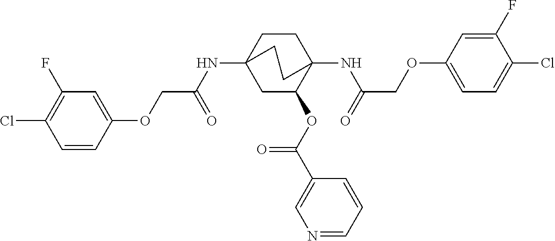 [SMILES: O=C(COc1ccc(Cl)c(F)c1)NC12CCC(NC(=O)COc3ccc(Cl)c(F)c3)(CC1)[C@@H](OC(=O)c1cccnc1)C2] |
| 129 | 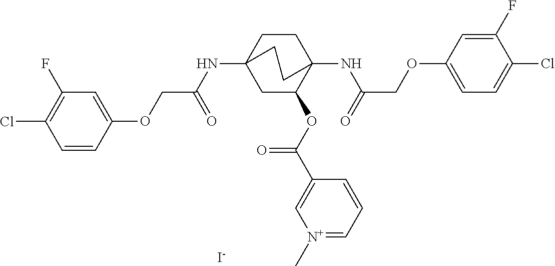 [SMILES: C[n+]1cccc(C(=O)O[C@H]2CC3(NC(=O)COc4ccc(Cl)c(F)c4)CCC2(NC(=O)COc2ccc(Cl)c(F)c2)CC3)c1 ; [I-]] |
| 130 | 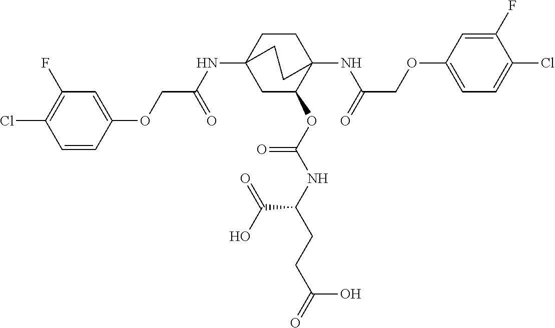 [SMILES: O=C(O)CC[C@@H](NC(=O)O[C@H]1CC2(NC(=O)COc3ccc(Cl)c(F)c3)CCC1(NC(=O)COc1ccc(Cl)c(F)c1)CC2)C(=O)O] |
| 131 | 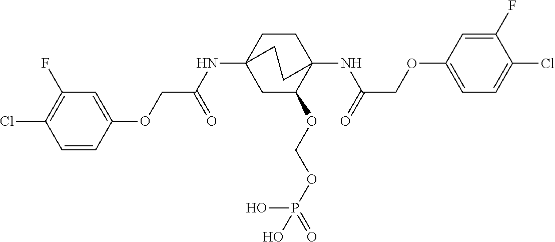 [SMILES: O=C(COc1ccc(Cl)c(F)c1)NC12CCC(NC(=O)COc3ccc(Cl)c(F)c3)(CC1)[C@@H](OCOP(=O)(O)O)C2] |
| 132 | 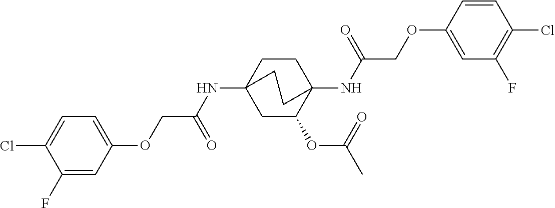 [SMILES: CC(=O)O[C@@H]1CC2(NC(=O)COc3ccc(Cl)c(F)c3)CCC1(NC(=O)COc1ccc(Cl)c(F)c1)CC2] |
| 133 | 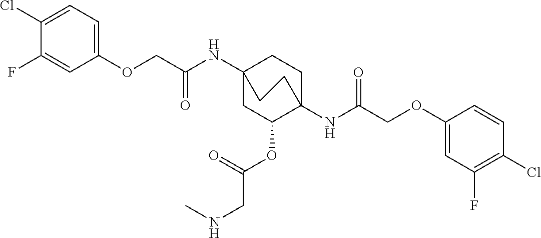 [SMILES: CNCC(=O)O[C@@H]1CC2(NC(=O)COc3ccc(Cl)c(F)c3)CCC1(NC(=O)COc1ccc(Cl)c(F)c1)CC2] |
| 134 | 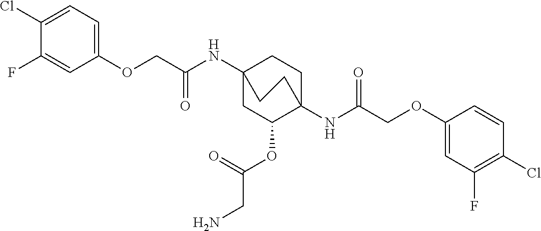 [SMILES: NCC(=O)O[C@@H]1CC2(NC(=O)COc3ccc(Cl)c(F)c3)CCC1(NC(=O)COc1ccc(Cl)c(F)c1)CC2] |
| 135 | 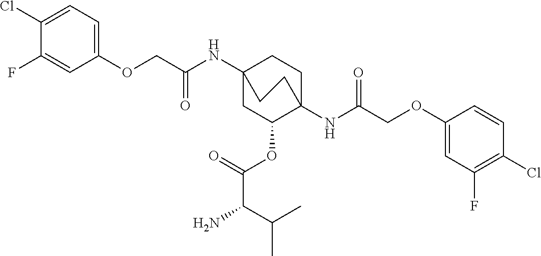 [SMILES: CC(C)[C@H](N)C(=O)O[C@@H]1CC2(NC(=O)COc3ccc(Cl)c(F)c3)CCC1(NC(=O)COc1ccc(Cl)c(F)c1)CC2] |
| 136 | 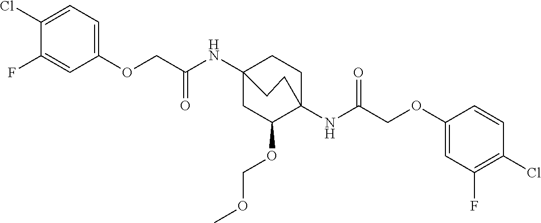 [SMILES: COCO[C@H]1CC2(NC(=O)COc3ccc(Cl)c(F)c3)CCC1(NC(=O)COc1ccc(Cl)c(F)c1)CC2] |
| 137 | 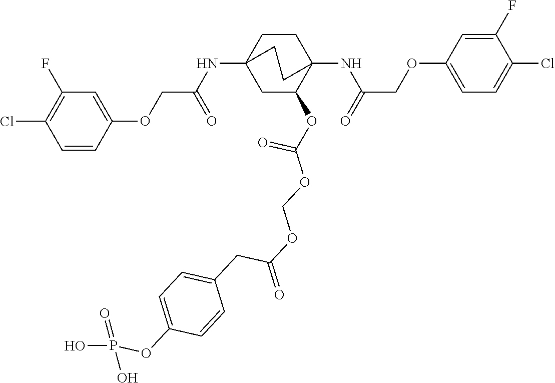 [SMILES: O=C(COc1ccc(Cl)c(F)c1)NC12CCC(NC(=O)COc3ccc(Cl)c(F)c3)(CC1)[C@@H](OC(=O)OCOC(=O)Cc1ccc(OP(=O)(O)O)cc1)C2] |
| 138 | 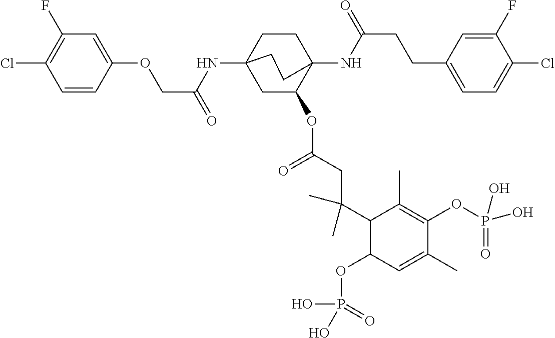 [SMILES: CC1=CC(OP(=O)(O)O)C(C(C)(C)CC(=O)O[C@H]2CC3(NC(=O)COc4ccc(Cl)c(F)c4)CCC2(NC(=O)CCc2ccc(Cl)c(F)c2)CC3)C(C)=C1OP(=O)(O)O] |
| 139 | 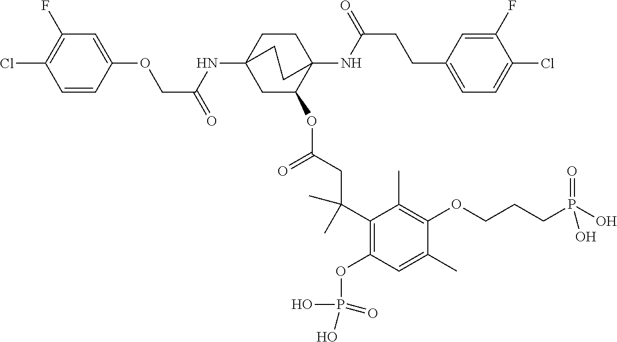 [SMILES: Cc1cc(OP(=O)(O)O)c(C(C)(C)CC(=O)O[C@H]2CC3(NC(=O)COc4ccc(Cl)c(F)c4)CCC2(NC(=O)CCc2ccc(Cl)c(F)c2)CC3)c(C)c1OCCCP(=O)(O)O] |
| 140 | 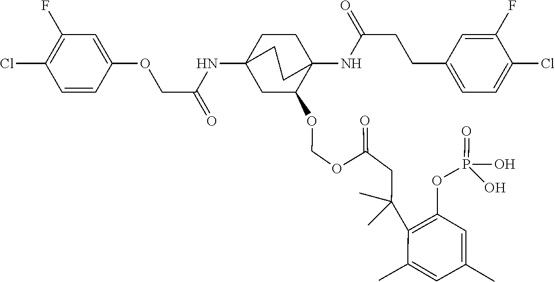 [SMILES: Cc1cc(C)c(C(C)(C)CC(=O)OCO[C@H]2CC3(NC(=O)COc4ccc(Cl)c(F)c4)CCC2(NC(=O)CCc2ccc(Cl)c(F)c2)CC3)c(OP(=O)(O)O)c1] |
| 141 | 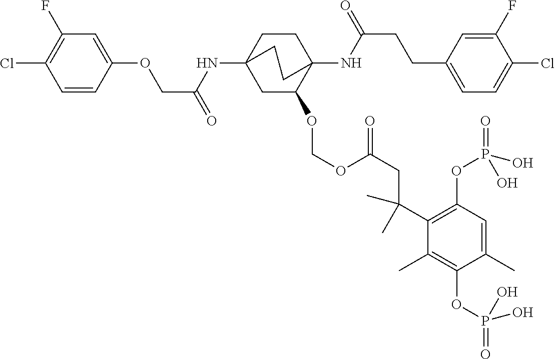 [SMILES: Cc1cc(OP(=O)(O)O)c(C(C)(C)CC(=O)OCO[C@H]2CC3(NC(=O)COc4ccc(Cl)c(F)c4)CCC2(NC(=O)CCc2ccc(Cl)c(F)c2)CC3)c(C)c1OP(=O)(O)O] |

### Pharmaceutical Compositions and Routes of Administration

Compounds provided in accordance with the present disclosure are usually administered in the form of pharmaceutical compositions. This disclosure therefore provides pharmaceutical compositions that contain, as the active ingredient, one or more of the compounds described herein, or a pharmaceutically acceptable salt thereof, and one or more pharmaceutically acceptable excipients or carriers. In some embodiments, the compound (e.g., a compound of Formula (I)) is provided in an effective amount in the pharmaceutical composition.

### EXAMPLES

The invention is further described in detail by reference to the following examples but are not intended to be limited to the following examples. These examples encompass any and all variations of the illustrations with the intention of providing those of ordinary skill in the art with complete disclosure and description of how to make and use the subject invention and are not intended to limit the scope of what is regarded as the invention.

### Example 1. GDF15 is Elevated in Cerebrospinal Fluid but not in Plasma in Vanishing White Matter Disease Model Mice

To characterize GDF15 under conditions of chronic ISR in the central nervous system (CNS), a vanishing white matter (VWMD) model was used. VWMD is a severe leukodystrophy of the CNS caused by mutations in any of the 5 genes encoding subunits of eIF2B and represents a model of chronic ISR activation in CNS.

In this example, a VWMD mouse model harboring a R191H mutation in eIF2B was used. These VWMD mice were previously characterized and were shown to develop persistent ISR that is confined to the CNS evident already at 2 months of age, resulting in a marked loss of white matter, and accompanied by a gradual loss of motor function starting at 5 months of age.

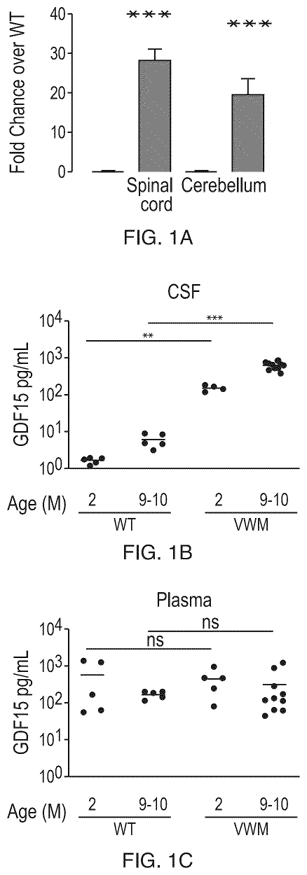

GDF15 gene expression in the cerebellum and spinal cord of VWM mice and age-matched WT mice were determined. FIG. 1A shows mean GDF15 levels were elevated 20- and 30-fold in the cerebellum and spinal cord of VWM mice as compared to WT mice, respectively.

GDF15 protein level was also evaluated. FIG. 1B and FIG. 1C show cerebrospinal fluid (CSF) levels of GDF15 were elevated more than 80-fold in 9-10 months-old VWM mice as compared to age-matched WT mice. Further, high levels of CSF GDF15 were detected at 2-month-old VWM mice and reached a maximum level with the initiation of motor dysfunction between 2 and 5 months of age. In contrast, matched plasma samples showed no difference in GDF15 levels between VWM and WT mice and across different ages.

### Example 2. Treatment with ISR Modulator Inhibits CSF GDF15 in VWM Mice

Effects of ISR modulator on GDF15 level were evaluated using ISR modulator 2BAct. 2BAct was previously shown to suppress the ISR signature genes, including Atf5, Eif4ebp1 and Trib3, in the brain and corrected motor deficit.

VWM mice and WT mice were treated with 2BAct at 30 mg/kg for four months in food pellets. Subsequently, expressions of ISR signature genes and GDF15 in the cerebellum were determined.

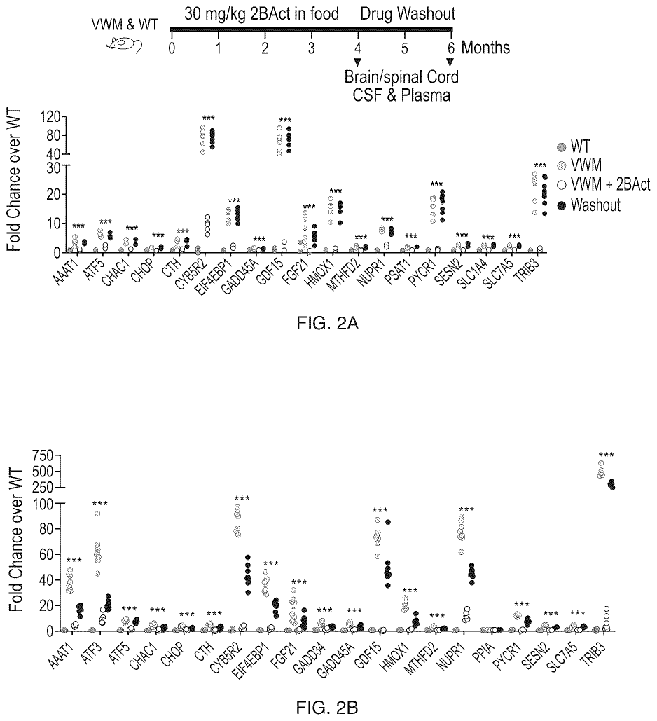

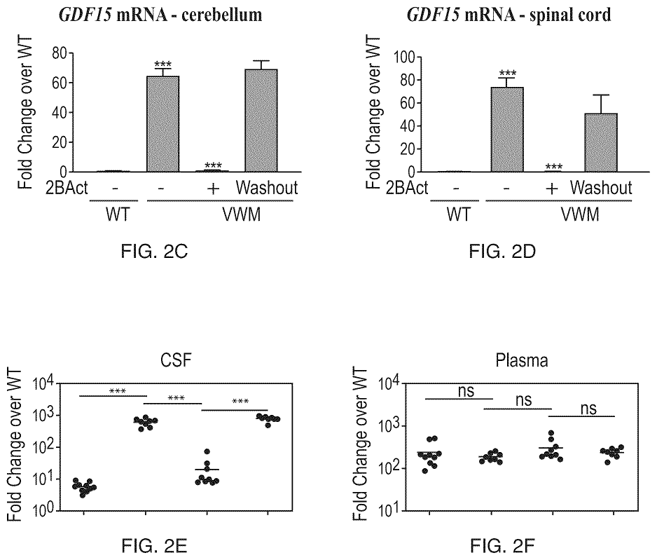

Exemplary results are shown in FIGS. 2A-2C. Treatment of VWM mice with 30 mg/kg 2BAct for four months inhibited the ISR gene expression signature in the cerebellum (FIG. 2A) and the spinal cord (FIG. 2B). GDF15 mRNA levels of in the CNS of VWM mice were markedly reduced following 2BAct treatment and rebounded to pre-treatment levels 2 months after cessation of 2BAct treatment (FIG. 2C and FIG. 2D). Similarly, CSF GDF15 levels were suppressed after 4-month treatment with 2BAct and returned to pre-dose baseline levels following 2 months of drug washout (FIG. 2E). In contrast, 2BAct treatment did not affect plasma GDF15 in VWM mice (FIG. 2F).

### Example 3. GDF15 Expression is Elevated in Astrocytes of VWM Mice

To investigate the potential cellular source of GDF15 in the CNS, single-cell RNA sequencing (scRNA-seq) was utilized to analyze the forebrain area of VWM mice. Briefly, forebrain were dissected from 2-month-old or 5-month-old female mice. Tissues were dissociated and prepared as single cell suspension. Single-cell RNA-seq libraries were prepared following manufacturer's instructions (10× Genomics Chromium), and cDNAs were amplified and sequenced. Obtained data were analyzed using bioinformatics and computational methods.

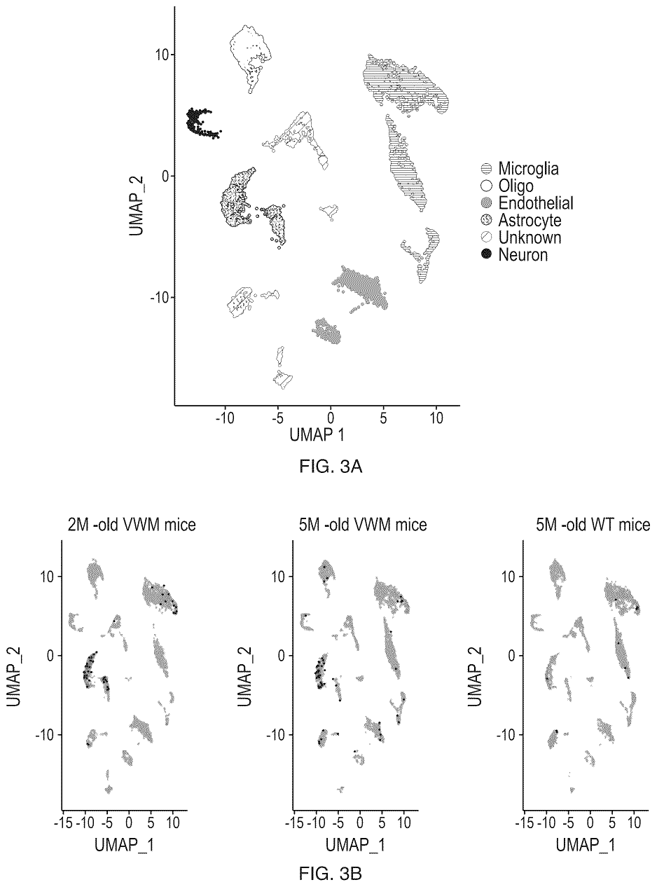

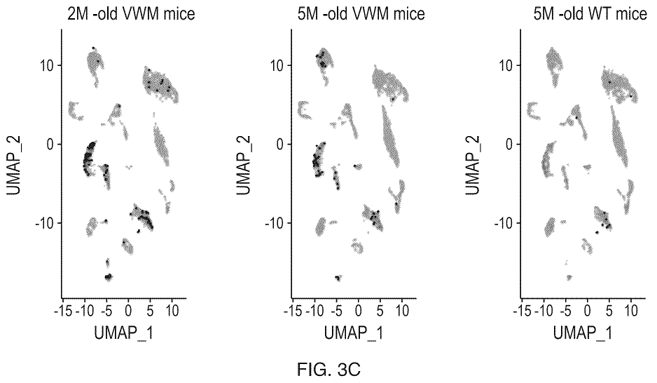

Exemplary results are shown in FIGS. 3A-3C. In 2-month old pre-symptomatic VWM mice, the bulk majority of GDF15 mapped to astrocytes (FIG. 3B). At 5-month old, GDF15 expression in astrocytes was evident with additional, yet minimal contribution from microglia and oligodendrocytes. No appreciable expression of GDF15 was detected in 5-month-old WT mice. As a positive control, TRIB3 (a representative ISR gene) expression was also prevalent in astrocytes as well as in endothelial cells (FIG. 3C). These data suggest astrocytes as the primary source of GDF15 in the VWM brain and CSF.

### Example 4. GDF15 is Elevated in CSF from VWMD Patients

To further characterize GDF15 under conditions of chronic ISR in the CNS, GDF15 levels in the CSF (n=6) as well as serum and plasma (n=16) samples from VWMD patients harboring different eIF2B loss-of-function (LOF) mutations (Table 2) were examined and compared to healthy volunteers.

TABLE 2
Patient Samples

| | | | | Genotype | | |
|---|---|---|---|---|---|---|
| Donor | | Age at | | eIFB | Allele 1 (nucleotide/ | Allele 1 (nucleotide/ |
| ID # | Gender | Sampling | Matrix | Subunit | amino acid | amino acid |
| 1 | F | 29 | CSF | 2 | C512T/S171F | 607-612 > del/insTG: |
| | | 39 | CSF | | | M203fs |
| | | 29 | Plasma | | | |
| | | 35 | Serum | | | |
| | | 39 | Serum | | | |
| 2 | F | 24 | Plasma | 4 | C1393T/C465R | C1465T/Y489H |
| 3 | F | 19 | Plasma | 3 | C260T/A87V | G455T |
| 4 | F | 23 | Plasma | 4 | C1393T/C465R | C1465T/Y489H |
| 5 | F | 16 | Plasma | 5 | A638G/E213G | G910G/E304stop |
| | | 25 | Plasma | | | |
| 6 | M | 21 | Serum | 2 | A638G/E213G | G910G/E304stop |
| 7 | M | 20 | CSF | 5 | G338A/R113H | G338A/R113H |
| | | | Serum | | | |
| 8 | M | 13 | Serum | 5 | G338A/R113H | T218G/V73G |
| 9 | F | 16 | Plasma | 5 | G338A/R113H | C583T/R195C |
| 10 | F | 9 | Serum | 5 | G338A/R113H | C1264T/R422STOP |
| | | 15 | Plasma | | | |
| 11 | F | 4 | CSF | 5 | G338A/R113H | G1888A/W628STOP |
| 12 | F | 4 | Serum | 5 | G338A/R113H | C0855T/R269X |
| 13 | M | 10 | Plasma | 5 | G338A/R113H | T218G/V73G |
| | | 13 | Plasma | | | |
| 14 | M | 13 | CSF | 4 | C1069T/R357W | C1069T/R357W |
| | | | Serum | | | |
| 15 | F | 12 | CSF | 5 | A1028G/Y343C | A1153G/I385V |
| | | | Plasma | | | |
| 16 | F | 6 | Plasma | 5 | G338A/R113H | C47A/A16D |
| 17 | M | 8 | CSF | 3 | G338A/R113H | G806A/R269L |
| 18 | M | 5 | Plasma | 5 | G338A/R113H | C47A/A16D |
| | | 6 | Plasma | | | |

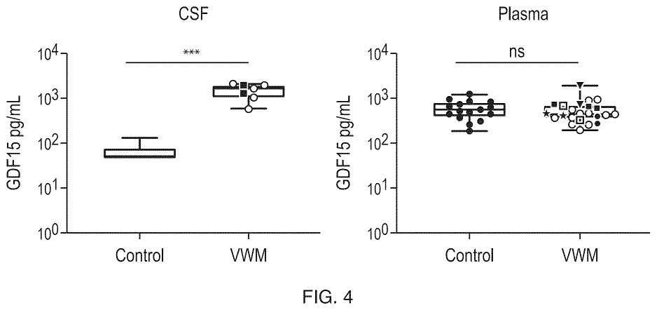

Mean GDF15 were, on average, 23-fold higher in CSF of VWMD patients, as compared to CSF from healthy controls (FIG. 4). Marked elevation in CSF GDF15 was observed in all 6 VWMD patients tested regardless of the type of mutation or age of sampling. In contrast, no significant difference of GDF15 was observed in the plasma or serum of VWMD patients as compared to healthy controls.

These data further corroborate the observation in VWM mouse model and suggest that the elevated levels of GDF15 in CSF, but not plasma or serum, in VWMD patients are likely to be associated with persistent activation of the ISR in the CNS.

### Example 5. GDF15 Expression in Neuron-Derived EVs of VWM

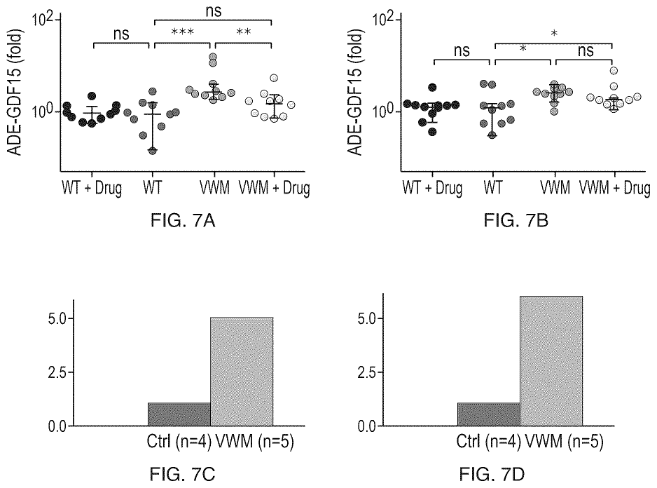

GDF15 protein was quantified in astrocyte-derived exosomes (ADEs) or neuron-derived exosomes (NDEs) in plasma using immunoassay. ADEs and NDEs were captured with anti-GLAST antibody and anti-SNAP25 antibody, respectively. GLAST is an astrocyte specific cell-surface protein; SNAP25 is highly expressed on neuronal membrane. The detection of GDF15 was performed by incubation with a specific anti-GDF15 antibody followed by an anti-mouse IgG conjugated to Horseradish peroxidase. GDF15 was significantly elevated in ADEs (FIG. 7A) and NDEs (FIG. 7B) from VWM as compared to WT mice. Treatment with 30 mg/kg 2Bact for 10 days suppressed GDF15 expression in plasma ADE and NDE to the level present in WT mice (FIGS. 7A and 7B).

GDF15 levels were also analyzed in ADEs and NDEs in plasma from VWM patients and compared to that of healthy subjects. GDF15 levels are 5-fold and 6.2-fold higher in plasma ADE and plasma NDE, respectively, from VWM patients as compared to healthy controls (FIGS. 7C and 7D).

These data suggest that GDF15 in ADE or NDE in plasma but not direct total plasma can be used to determine the ISR in the CNS.

### Example 6. CSF GDF15 is Elevated in G93A SOD1 Mice and ALS Patients

ISR signature and GDF15 expression were evaluated in SOD1 G93A mouse model and samples from ALS (amyotrophic lateral sclerosis) patients.

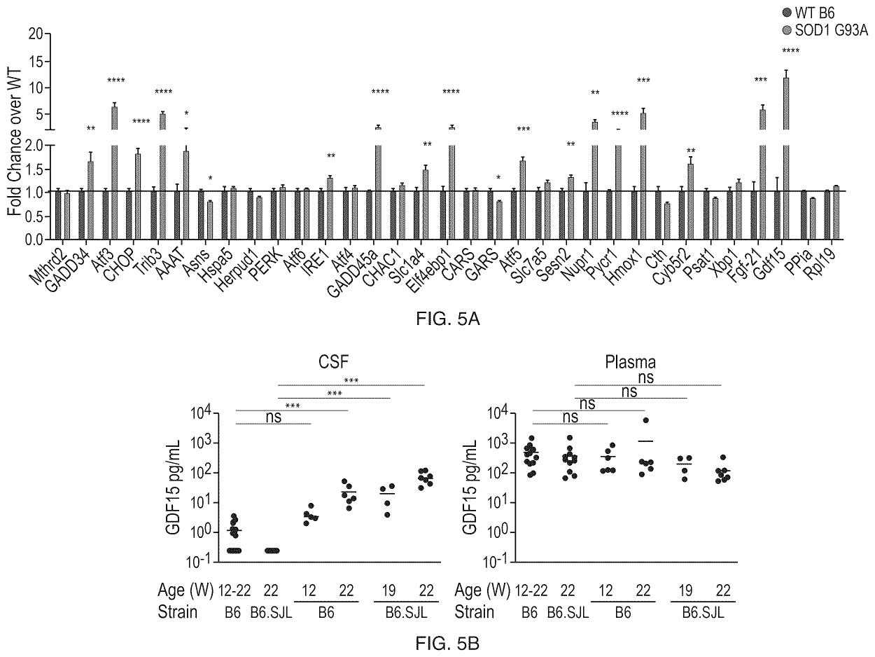

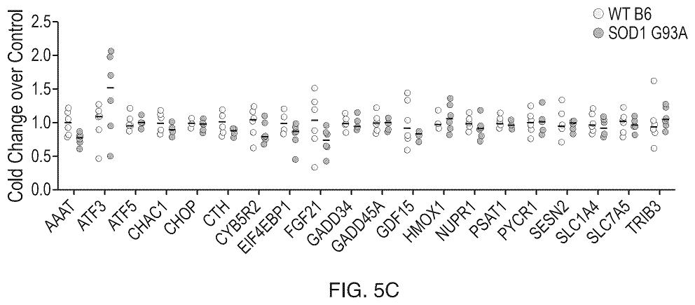

Gene expression analysis of the spinal cord of the SOD1 G93A mice reveled elevated ISR signature as compared to WT mice, as well as the GDF15 mRNA (FIG. 5A). GDF15 protein expression level was also significantly elevated in the CSF of the SOD1 G93A mice as compared with WT (FIG. 5B). Similar to in VWM mice, such elevation was not detected in plasma. The elevated GDF15 was not observed in the cerebellum, implicating the spinal cord as the primary source for GDF15 in the CSF (FIG. 5C).

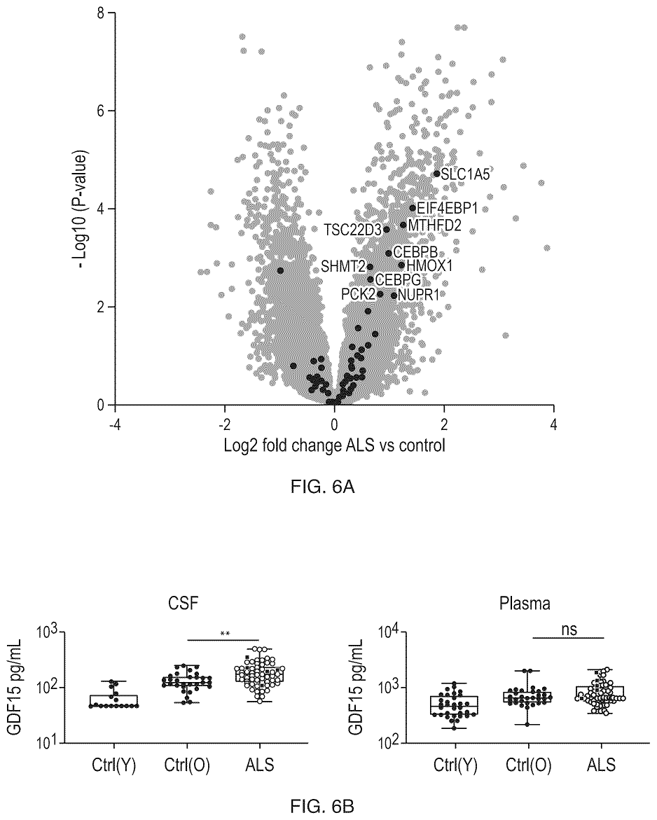

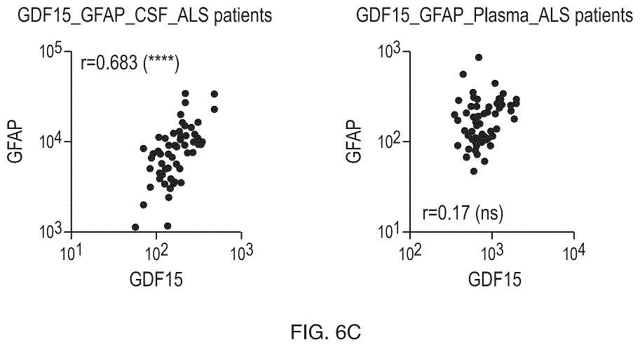

In samples from ALS patients, gene expression analysis in spinal cord demonstrated that ISR gene expression signature is elevated in these patients as compared with healthy subjects (ALS, n=104; healthy controls, n=22) (FIG. 6A). CSF and blood samples were analyzed from ALS patients (n=54) and age-matched (n=29 CSF, n=31 plasma; mean age 62) and young (n=16; mean age 23.5) controls. GDF15 was significantly elevated in CSF from ALS patients (203.0 pg/mL) as compared to age-matched controls (131.6 pg/mL) while no elevation was registered in plasma levels (FIG. 6B). Age-related increase of GDF15 in the CSF was evident in both healthy and ALS patients but mean levels were overall higher in ALS. It should be noted that the distribution of CSF GDF15 levels in ALS patients overlapped with healthy range levels, indicating large inter-patient variability of this biomarker in ALS.

To further assess the cellular origin of GDF15, several cell-specific biomarkers in CSF and plasma were evaluated. Levels of GFAP, a marker of astrocyte activation, were highly correlated to CSF GDF15 levels in ALS samples (FIG. 6C).

These data suggest that ISR is elevated in the spinal cord in ALS pathology and is associated with increased CSF GDF15 level, possibly of astrocytic-source.

### EQUIVALENTS/OTHER EMBODIMENTS

Those skilled in the art will recognize or be able to ascertain using no more than routine experimentation, many equivalents to the specific embodiments described herein. Such equivalents are intended to be encompassed by the following claims.

What is claimed is:

1. A method of treating a neurodegenerative condition, the method comprising:
determining the GDF15 expression level in a sample obtained from a patient;
comparing the GDF15 expression level in the sample to a reference;
determining that the GDF15 expression level in the sample is elevated as compared to the reference; and
administering an integrated stress response (ISR) modulator.

2. A method of treating a neurodegenerative condition in a patient comprising administering to the patient an integrated stress response (ISR) modulator, wherein GDF15 expression level is elevated in a sample obtained from the patient prior to administration of the ISR modulator as compared to a reference.

3. A method of treating a patient having integrated stress response dysregulation, the method comprising administering an integrated stress response (ISR) modulator to the patient, wherein GDF15 expression level in a sample obtained from the patient is elevated as compared to a reference.

4. The method of any one of claims 1-3, wherein the reference is determined by measuring the GDF15 expression level in a sample obtained from an aged match healthy subject.

5. A method of evaluating a compound to determine whether the compound can modulate the integrated stress response, the method comprising:
administering the compound to a subject;
measuring GDF15 expression level in a sample obtained from the subject; and
comparing the GDF15 expression level to a reference, wherein a significant difference between the GDF15 expression level and the reference is indicative that the compound can modulate the integrated stress response.

6. The method of claim 5, wherein the reference is determined by measuring the GDF15 expression level in a sample obtained from the subject before administering the compound.

7. The method of any one of claims 1-6, wherein the sample is a fluid sample.

8. The method of claim 7, wherein the fluid sample is a cerebrospinal fluid (CSF) sample.

9. The method of claim 7, wherein the fluid sample is a blood sample.

10. The method of claim 9, wherein the blood sample is enriched for central nervous system (CNS)-derived extracellular vesicles (e.g., exosomes).

11. The method of claim 10, wherein the CNS-derived extracellular vesicles are astrocyte-derived exosomes (ADEs).

12. The method of claim 10, wherein the CNS-derived extracellular vesicles are neuron-derived exosomes (NDEs).

13. The method of any one of claims 1-12, wherein the GDF15 expression level is mRNA level or protein level.

14. The method of any one of claims 1-13, wherein the GDF15 expression level is significantly higher than the reference.

15. The method of any one of claims 1-14, wherein the GDF15 expression level is at least about 10%, at least about 20%, at least about 30%, at least about 40%, at least about 60%, or at least about 80% higher than the reference.

16. The method of any one of claims 1-15, wherein the ISR modulator is an eIF2B activator.

17. The method of any one of claims 1-16, wherein the ISR modulator is a compound selected from Table 1, or a pharmaceutically acceptable salt thereof.
